(19) United States
(12) Patent Application Publication
(10) Pub. No.: US 2004/0131574 A1
(43) Pub. Date: Jul. 8, 2004

(54) Method and composition for the control of hair growth

(76) Inventor: Raymond J Bergeron JR., Gainesville, FL (US)

Correspondence Address: Dennis P Clarke, Miles & Stockbridge, Suite 500, 1751 Pinnacle Drive, McLean, VA, 22102-3833, US

(21) Appl. No.: 10/467,366

(22) PCT Filed: Feb. 7, 2002
(86) PCT No.: PCT/US02/03363

(30) Foreign Application Priority Data
Feb. 7, 2001 (US) 60266871

(51) Int. Cl. A61K 7/06; A61K 7/11

(52) U.S. Cl. 424/70.17

(57) ABSTRACT

Disclosed are pharmaceutical compositions and methods for controlling the growth of hair by topically applying to a human or non-human mammal a polyamine or a salt thereof with a pharmaceutically acceptable acid, the polyamine having formula (I) wherein: R₁-R₆ may be the same or different and are hydrogen, alkyl, hydrocarbyl aryl, hydrocarbyl aryl alkyl, cycloalkyl, or any of the foregoing wherein the alkyl chain is interrupted by at least one etheric oxygen atom with the proviso that R₁ and R₆ may not both be hydrogen; N¹, N², N³ and N⁴ are nitrogen atoms capable of protonation at physiological pHs; a and b may be the same or different and are integers from 0 to 6; A, B and C may be the same or different and are integers from 0 to 6; A, B and C may be the same or different and are bridging groups of variable length, including unsubstituted heterocyclic bridging groups which incorporate as a hetero atom therein said N¹ or N⁴; and further wherein said A, B, C are selected such that they effectively maintain the distance between the nitrogen atoms such that the polyamine: (i) is capable of uptake by a target cell upon topical application of the polyamine to said human or non-human mammal; and (ii) upon uptake by the target cell, competitively binds via an electrostatic interaction between the positively charged nitrogen atoms to substantially the same biological counter-anions as the intracellular natural polyamines in the target cell; the polyamine, upon binding to the biological counter-anion in the cell, functions to inhibit the formation therein of the enzyme, ornithine decarboxylase.

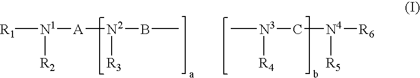

### BACKGROUND OF THE INVENTION

1. Field of the Invention

The present invention relates to control of growth of unwanted hair.

2. Description of the Prior Art

In recent years, a great deal of attention has been focused on the polyamines, e.g., spermidine, norspermidine, homospermidine, 1,4-diaminobutane (putrescine) and spermine. These studies have been largely directed at the biological properties of the polyamines probably because of the role they play in proliferative processes. It was shown early on that the polyamine levels in dividing cells. e.g., cancer cells, are much higher than in resting cells. See Janne et al, A. Biochem. Biophys. Acta., Vol. 473, page 241 (1978); Fillingame et al, Proc. Natl. Acad. Sci. U.S.A. Vol. 72. page 4042 (1975); Metcalf et al. J. Am. Chem. Soc., Vol. 100, page 2551 (1978): Flink et al. Nature (London), Vol. 253. page 62 (1975); and Pegg et al, Polyamine Metabolism and Function, Am. J. Cell. Physiol., Vol. 243, pages 212-221 (1982).

Several lines of evidence indicate that polyamines, particularly spermidine, are required for cell proliferation: (i) they are found in greater amounts in growing than in non-growing tissues; (ii) prokaryotic and eukaryotic mutants deficient in polyamine biosynthesis are auxotrophic for polyamines; and (iii) inhibitors specific for polyamine bio-synthesis also inhibit cell growth. Despite this evidence, the precise biological role of polyamines in cell proliferation is uncertain. It has been suggested that polyamines, by virtue of their charged nature under physiological conditions and their conformational flexibility, might serve to stabilize macromolecules such as nucleic acids by anion neutralization. See Dkystra et al, Science. Vol. 149, page 48 (1965); Russell et al, Polyamines as Biochemical Markers of Normal and Malignant Growth (Raven, New York. 1978); Hirschfield et al, J. Bacteriol., Vol. 101, page 725 (1970); Hafner et al, J. Biol. Chem., Vol. 254, page 12419 (1979); Cohn et al, J. Bacteriol., Vol. 134, page 208 (1978); PohJatipelto et al. Nature (London). Vol. 293, page 475 (1981); 40 Mamont et al, Biochem. Biophys. Res. Commun., Vol. 81, page 58 (1978); Bloomfield et al, Polyamines in Biology and Medicine (D. R. Morris and L. J. Morton, eds., Dekker, New York, 1981), pages 183-205; Gosule et al, Nature, Vol. 259, page 333 (1976); Gabbay et al, Ann. N.Y. Acad. Sci. Vol. 17 1. page 810 (1970); Suwalsky et al, J. Mal. Biol., Vol. 42, page 363 (1969); and Liquori et al, J. Mol. Biol., Vol. 24, page 113 (1968).

However, regardless of the reason for increased polyamine levels, the phenomenon can be and has been exploited in chemotherapy. See Sjoerdsma et al, Butterworths Int. Med. Rev.: Clin. Pharmacol. Thera., Vol. 35, page 287 (1984); Israel et al, J. Med. Chem., Vol. 16. page 1 (1973); Morris et al, Polyamines in Biology and Medicine, Dekker. New York, page 223 (1981); and Wang et al, Biochem. Biophys. Res. Commun. Vol. 94, page 85 (1980).

Because of the role the natural polyamines play in proliferation, a great deal of effort has been invested in the development of polyamine analogues as anti-proliferatives [Cancer Res., Vol. 49, “Role of the methylene backbone in the anti-proliferative activity of polyamine analogues on L1210 cells,” Bergeron et al, pages 2959-2964 (1989); J. Med. Chem. Vol. 31, “Synthetic polyamine analogues as antineoplastics,” Bergeron et al, pages 1183-1190 (1988); Polyamines in Biochemical and Clinical Research, “Regulation of polyamine biosynthetic activity by spermidine and spermine-a novel antiproliferative strategy,” Porter et al, pages 677-690 (1988); Cancer Res., Vol. 49, “Major increases in spermidine/spermine-N¹-acetyl transferase activity by spermine analogues and their relationship to polyamine depletion and growth inhibition in L1210 cells,” Basu et al, pages 6226-6231 (1989); Biochem. J. Vol. 267, “Induction of spermidine/spermine N¹-acetyltransferase activity in Chinese-hamster ovary cells by N¹,N¹¹-bis(ethyl) nonspermine and related compounds,” Pegg et al. pages 331-338 (1990); Biochem. J. Vol. 268. “Combined regulation of ornithine and S-adenosylmethionine decarboxylases by spermine and the spermine analogue N¹N¹²-bis(ethyl) spermine.” Porter et al, pages 207-212 (1990); Cancer Res., Vol. 50, “Effect of N¹, N¹⁴-bis(ethyl)-homospermine on the growth of U-87 MG and SF-126 on human brain tumor cells,” Basu et al. pages 3137-3140 (1990); and Biochem. Biophys. Res. Commun., Vol. 152, “The effect of structural changes in a polyamine backbone on its DNA binding properties,” Stewart, pages 1441-1446 (1988)]. These efforts have included the design of new synthetic methods[i. Org. Chem., Vol. 45, “Synthesis of N⁴-acylated N¹,N⁸bis(acyl)spermidines: An approach to the synthesis of siderophores,” Bergeron et al. pages 1589-1592 (1980); Synthesis, “Reagents for the selective acylation of spermidine, homospermidine and bis-[13-amino]propyl amine.” Bergeron et al, pages 732-733 (1981); Synthesis, “Reagents for the selective secondary functionalization of linear trianines,” Bergeron et al, pages 689-692 (1982); Synthesis, “Amines and polyamines from nitriles.” Bergeron et al. pages 782-785 (1984); J. Org. Chem. Vol. 49, “Reagents for the stepwise functionalization of spermidine, homospermidine and bis-[13-aminopropyl]amine.” Bergeron et al, page 2997 (1984); Accts. Chem. Res., Vol. 19. “Methods for the selective modification of spermidine and its homologues,” Bergeron, pages 105-113 (1986); Bioorg. Chem., Vol. 14, “Hexahydroxypyrimidines as masked spermidine vectors in drug delivery.” Bergeron et al, pages 345-355 (1986); J. Org. Chem., Vol. 53. “Reagents for the stepwise functionalization of spermine,” Bergeron et al, pages 3108-3111 (1988); J. Org. Chem., Vol. 52, “Total synthesis of (±)-15-Deoxyspergualin,” Bergeron et al, pages 1700-1703 (1987); J. Org. Chem., Vol. 56, “The total synthesis of Alcaligin.” Bergeron et al, pages 586-593 (1991); and CRC Handbook on Microbial Iron Chelates, “Synthesis of catecholamide and hydroxamate siderophores,” Bergeron et al. pages 271-307 (1991)] for the production of these analogues, as well as extensive biochemical studies focused on the mechanism by which these compounds act [Cancer Res., Vol. 46, “A comparison and characterization of growth inhibition in L1210 cells by (2-Difluoromethylomithine (DFMO), an inhibitor of ornithine decarboxylase and N¹,N⁸-bis(ethyl)spermidine (BES), an apparent regulator of the enzyme,” Porter et al, pages 6279-6285 (1986); Cancer Res., Vol. 47, “Relative abilities of bis(ethyl) derivatives of putrescine, spermidine and spermine to regulate polyamine biosynthesis and inhibit L1210 leukemia cell growth,” Porter et al, pages 2821-2825 (1987); Cancer Res., Vol. 49, “Correlation between the effects of polyamine analogues on DNA conformation and cell growth,” Basu et al. pages 5591-5597 (1989); Cancer Res., Vol. 49, “Differential response to treatment with the bis(ethyl)polyamine analogues between human small cell lung carcinoma and undifferentiated large cell lung carcinoma in culture.” Casero et al, pages 639-643 (1988); Mol. Pharm., Vol. 39, “Selective cellular depletion of mitochondrial DNA by the polyamine analog, N¹,N¹²-bis(ethyl)spermine, and its relationship to polyamine structure and function,” Vertino et al, pages 487-494 (1991); Biochem. and Biophys. Res. Comm., Vol. 157. “Modulation of polyamine biosynthesis and transport by oncogene transfection,” Chang et al, pages 264-270 (1988); and Biopolymers, Vol. 26, “Structural determinants of spermidine-DNA interactions,” Vertino et al, pages 691-70-3 (1987). The mechanistic investigations have encompassed uptake studies, impact on polyamine analogues on polyamine pools and polyamine biosynthetic enzymes, as well as their effects on translational and transcriptional events.

Anti-neoplastic analogues of the naturally occurring polyamines, pharmaceutical compositions and methods of treatment are also disclosed in the following pending patent application Ser. No. 08/231,692 filed Apr. 25, 1994, as well as in U.S. Pat. Nos. 5,091,576 issued Feb. 25, 1992; 5,128,353 issued Jul. 7, 1992; and 5,173,505 issued Dec. 22, 1992. The disclosures of each of the foregoing applications and patents are incorporated herein by reference.

Intensive medical investigation has been directed to the elucidation of the mechanism of hair growth and the role of the endocrine system therein. As a result of such investigations, it is generally agreed that the fine, light-colored vellus hair, which covers most of the body during childhood, comes under the influence of growth hormones and of androgens to eventually become the coarser and darker terminal hairs which characterize many areas of the adult body. The desire to discover methods for controlling androgen-dependent conditions has generated a large number of studies dealing with androgen metabolism in skin. These studies have suggested that it is possible to reduce the amount of androgen capable of entering into the hair growth cycle by two routes.

First, the conversion of serum testosterone to dihydrotestosterone can be prevented by the inhibition of the enzyme steroid 5-alpha-reductase. Secondly, certain compounds can compete with the testosterone or dehydrotestosterone for the cytoplasmic receptor sites. The action of both types of anti-androgen compounds in skin can also affect the course of male-pattern hair growth in females, thus leading to their application in the treatment of female hirsutism. Such application is described, inter alia, in the following patents:

U.S. Pat. Nos. 4,139,638 and 4,151,540 describe the use of 4′-substituted and certain 3′,4′-disubstituted anilides for the treatment of androgen-dependent disease states such as female hirsutism and acne.

U.S. Pat. No. 4,191,775 discloses that certain disubstituted- branched-chain-fluorinated-acylanilides may be used in the topical treatment of androgen-dependent disease conditions such as acne, female hirsutism, and seborrhea.

U.S. Pat. No. 4,344,943 describes the topical use of certain androgenic 17-alpha-substituted steroids exemplified by 17-beta-hydroxyl-1-alpha-methyl-17-alpha. (1-methyl-2-propenyl)-5-alpha-androstan-3-one for the treatment of diseases such as acne, seborrhea, alopecia and female hirsutism.

West German OLS No. 2,940,14.4 describes the use of combinations of progesterone with either cyproterone acetate or chlormadinone acetate in the topical treatment of androgen-induced hormonal disturbances such as alopecia, female hirsutism, and acne.

The patent art also discloses a number of non-steroidal methods of reducing the growth of human hair as opposed to its conventional removal by cutting, shaving, or depilation. One such method is described in U.S. Pat. No. 3,426,137, which pertains to a process, for inhibiting the growth of hair by the topical application to a depilated skin area of a composition containing a substituted benzophenone such as 2-amino-5-chlorobenzophenone. Examples in the patent illustrate the reduction of hair growth on the back area of rabbits and on the arm of a male human subject.

Another process for extending the duration of depilation is described in U.S. Pat. No. 4,370,315. The process therein comprises the topical application of a composition containing a lipoxygenase along with linoleic acid or derivative thereof. The patent describes the application of such compositions to various body parts of female subjects in the majority of which the regrowth of hair was clearly perceptible only after six or more weeks.

Ornithine decarboxylase (ODC) was essentially unknown until the late 1960's. This enzyme remained in a state of relative obscurity until it was realized that its activity is the rate-determining step in the biosynthesis of polyamines which are produced by mammalian species. The application of this discovery has led to the administration of ODC inhibitors in the treatment of a variety of conditions. Prior art references describing such applications include, inter alia:

U.S. Pat. No. 4,413,141 relating to 2-difluoromethyl(2,5-diaminopentanoic) acid or its salts as contra-gestational agents, for use in the treatment of benign prostatic hypertrophy, for use in slowing neoplastic cell proliferation and as an anti-protozoal agent.

U.S. Pat. No. 4,421,768 dealing with fluorinated diamino-heptene and -heptyne, derivatives for use in controlling the growth rate of rapidly proliferating tumor tissue and for controlling the growth of pathogenic parasitic protozoa.

U.S. Pat. No, 4,207,315 claims a process for treating non-malignant proliferative skin disease by the application of diamines of aliphatic hydrocarbons or derivatives of ornithine in association with a pharmaceutical carrier.

U.S. Pat. No. 4,201,789 claims a process for treating nonmalignant proliferative skin diseases by the administration of a compound exemplified by methyl glyoxal bis-(guanyl hydrazone) in association with a pharmaceutical carrier.

U.S. Pat. No. 4,720,489 discloses and claims compositions and methods for the control of hair growth based on the discovery that the topical application of ODC inhibitors to the skin slows the growth of hair in the treated areas. The inhibitors function by irreversibly binding with the ODC present in the cell to prevent the ODC from catalyzing the decarboxylation of ornithine to putrescine, a first step in the biosynthesis of such polyamines as spermine and spermidine. The mechanism of inhibitory activity of ODC inhibitors by irreversibly binding to mammalian ODC thereby inactivating it is described by B. Metcalf et al., J. Am. Chem. Soc., 100, 2551 (1978), C. Danzin et al., Life Sciences, 24, 519 (1979), and N. Seiler et al., Enzyme-Activated Irreversible Inhibitors, N. Seiler et al., Editors, Elsevier/North Holland Biomedical Press, 1978, pages 55 to 71, Kallio et al., Biochem. J., 200, 69 (1981) and Hupe et al., Fed. Proc. (Abstract No.5282), 41, 1174 (1982).

It is an object of the invention to provide novel methods and compositions for the regulation of hair growth in human and nonhuman mammals.

### SUMMARY OF THE INVENTION

One embodiment of the invention relates to a pharmaceutical composition in unit dosage form adapted for topical application to a human or non-human mammal to control the growth of hair in the area where applied comprising an amount effective therefor of a polyamine or a salt thereof with a pharmaceutically acceptable acid, the polyamine having the formula:

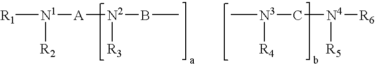

wherein:

R₁-R₆ may be the same or different and are hydrogen, alkyl, hydrocarbyl aryl, hydrocarbyl aryl allyl, cycloalkyl, or any of the foregoing wherein the alkyl chain is interrupted by at least one etheric oxygen atom with the proviso that R₁ and R₆ may not both be hydrogen;

N¹, N², N³ and N⁴ are nitrogen atoms capable of protonation at physiological pHs;

a and b may be the same or different and are integers from 0 to 6;

A, B and C may be the same or different and are bridging groups of variable length, including unsubstituted heterocyclic bridging groups which incorporate as a hetero atom therein the N¹ or N⁴;

and further wherein the A, B and C are selected such that they effectively maintain the distance between the nitrogen atoms such that the polyamine:

(i) is capable of uptake by a target cell upon topical application of the polyamine to the human or non-human mammal; and

(ii) upon uptake by the target cell, competitively binds via an electrostatic interaction between the positively charged nitrogen atoms to substantially the same biological counter-anions as the intracellular natural polyamines in the target cell; the polyamine, upon binding to the biological counter-anion in the cell, functions to inhibit the formation therein of the enzyme, ornithine decarboxylase.

A second embodiment of the invention concerns a method for controlling the growth of hair on a human or non-human mammal comprising topically applying to the mammal an effective amount of the polyamine described above.

### DETAILED DESCRIPTION OF THE INVENTION

As noted above, ODC inhibitors function by irreversibly binding with the ODC present in the cell to prevent the ODC from catalyzing the decarboxylation of ornithine to putrescine, a first step in the biosynthesis of such polyamines as spermine and spermidine. This is the mechanism by which the hair growth regulation formulas containing ODC inhibitors currently in use function. The present invention is predicated on the discovery that hair growth may be regulated by topically applying to human and nonhuman animals the polyamines described herein which prevent the formation of ODC in the first instance in the cells in which they are taken up. Thus, rather than functioning as “ODC inhibitors” as in the case of the prior art formulations, the polyamines of the invention function as “ODC synthesis preventers”.

The polyamines employed in the practice of the invention are any of those described in U.S. Pat. Nos. 5,342,945; 5,091,576; 5,962,533; 6,184,232; 6,147,262, and 5,866,613, the entire contents and disclosures of which are incorporated herein by reference.

In the polyamines of the invention, as described in the above formulas, R₁-R₆ may be alkyl, for example, methyl, ethyl, n-propyl, isopropyl, n-butyl, isobutyl, sec-butyl, tert-butyl and the like; hydrocarbyl aryl, for example, phenyl, p-tolyl, 2.4,6-trimethylphenyl and the like; hydrocarbyl aryl alkyl, for example, benzyl, α-phenethyl, β-phenethyl and the like; cycloalkyl, for example, cyclohexyl, cyclobutyl, cyclopentyl. cycloheptyl and the like; any of the foregoing wherein the alkyl chain is interrupted by etheric oxygen, for example, CH₃0(CH₂)₂—, CH₃0(CH₂)₂0(CH₂)₂—, CH₃0(CH₂)₂0(CH₂)₂0(CH₂)₂— and the like; or hydrogen.

At physiological pH's, the naturally occurring polyamines and the analogues of the present invention are largely in a protonated state [Bergeron et al., Bioorg. Chem., Vol. 14, pp. 345-355 (1986)]. Except where R₁-R₆ are hydrogen or etheric substituents, each R group is hydrocarbyl and may have from about 1 to about 12 carbon atoms, it being understood that the size of the substituents will be tailored in each case to ensure that the polyamine is capable of being protonated at a physiological pH.

The bridging groups of variable length, A, B and C may be the same or different and may be alkylene having 1-8 carbon atoms, for example, methylene, trimethylene, tetramethylene, pentamethylene and the like; branched alkylene, for example, —CH(CH)₃CH₂CH₂—, —CH₂CH(CH₃)CH₂—, —CH(CH₃)CH₂CH₂—, —CH₂CH(CH₃)CH₂CH₂— and the like; hydrocarbyl arylalkylene, for example, —CH(Ph)CH₂CH₂—, —CH₂CH(Ph)CH₂—, —CH(Ph)CH₂CH₂CH₂—, —CH₂CH(Ph)CH₂—CH₂— and the like; cycloalkylene, for example, cyclohexylene, cis- and trans- 1,2-cyclohexylene, 1,3-cyclohexylene, 1,4-cyclohexylene, 1,2-cyclopentylene, 1,3 cyclopentylene and the like; alkylene containing at least one —CH(OH)— group which is not alpha- to either of the nitrogen atoms; heterocyclic groups which incorporate within the ring one of the nitrogen atoms of the polyamine, it being understood that the heterocyclic nitrogen group may be located at the terminal end(s) or within the interior of the polyamine.

Polyamines of the present invention can possess one or more asymmetric carbon atoms and are thus capable of existing in the form of optical isomers, as well as in the form of racemic or non-racemic mixtures thereof. The preferable isomers are the (R,R) and (S,S) isomers, but also useful are the (R,S) and (S,R) isomers. The optical isomers can be obtained by stereospecific reactions utilizing optically active starting materials or by stereospecific resolution of the racemic mixtures according to processes known to those skilled in the art, for example, by formation of diastereoisomeric salts by treatment with an optically active acid or base. Examples of appropriate acids are tartaric, diacetyltartaric, dibenzoyltartaric, ditoluoyltartaric and camphorsulfonic acid and the like, followed by separation of the mixture of diastereoisomers by crystallization, then release of the optically active bases from these salts. Another example of a process for separation of optical isomers involves the use of a chiral chromatography column optimally chosen to maximize the separation of the enantiomers.

Most preferred among the polyamines for use in the methods and compositions of the invention are N,N′-diethylhomospermine, (R,R)-dihydroxydiethylhomospermine and (S,S)-dihydroxydiethylhomospermine.

Various compositions are provided herein for the administration of the agent of the invention as a topical composition, which in addition to the agent comprises a carrier or diluent for the agent, which is suitable for its transdermal delivery and optionally one or more of a variety of other ingredients suitable for inclusion therein. Examples of these ingredients are buffers, salt forming acids and bases, perfumes, colorants, emollients, adjuvants, single or multiple enteric coatings, copolymers, microporous or semipermeable membranes, enzyme inhibitors, mucoadhesives, chelating agents, particulate systems, viral envelope proteins, liposomes and other micelles, emulsifiers, lipoproteins and other fatty acid derivatives, surfactants, bile salts, hydrophilic, neutral, and hydrophobic polymers and co-polymers, hydrogels, biodegradable polymers and co-polymers or an exfoliant ingredient.

The composition may be in the form of a cream, an ointment, a solution, a gel, a powder, a suspension, an emulsion, encapsulated particles, or mixtures or combinations of these forms. Suitable pharmaceutically acceptable topical vehicles for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both volatile and non-volatile) such as cyclomethicone, demethiconol and dimethicone copolyol (Dow Coming); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art. The vehicle may further include components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The Extra Pharmacopoeia (Pharmaceutical Press, London 1993) and Martin (ed.), Remington's Pharmaceutical Sciences.

The agent may be present in different amounts, typically the dermal composition has about 0.1 to 25 wt % or more, and preferably about 5 to 25 wt %. However, other amounts larger and smaller may be suitable for special applications. Another preferred form of the composition is in the form of a controlled release composition wherein the formulation ingredients added control the rate of release of the agent. These may be degradable polymers and copolymers, matrixes which “leach out” the agent, and the like, as is known in the art. The composition may also be produced in the form of an implant for releasing a desired amount of the agent over a predetermined period of time. The composition of the invention may be provided as a kit with instructions for its use, particularly in terms of any necessary manipulations, the number and timing of the applications, and the area to be applied to, as well as the frequency taking into consideration different body surface area and weight of the subject. This is particularly important when it is applied or administered to children, and more importantly to infants and newborn babies, as well as to small animals. Smaller doses may be required in these cases. The composition may be delivered from a passive transdermal delivery device formed from a solid support with a compartment containing a solution or suspension comprising the composition. The compartment has a permeable side which is applied to an area of a subject's skin or dermis and the agent is allowed to pass from the device onto and through the skin, mucosal, or buccal surfaces of the subject. The device is preferably placed in a sealed sterile container immediately after manufacture by methods known in the art. A removable cover may be placed on the permeable side of the container prior to sealing and/or packaging to retain the solution or suspension of the agent during storage and prior to use.

The invention is illustrated by the following non-limiting examples.

### EXAMPLE 1

Fourteen C57/B16 mice were partially shaven; one was maintained as a control and to the remainder was administered diethyl-homospermine [10 mg/kg-0.01% solution (Bid)], six times daily over 9 days. The control resumed normal hair growth immediately in the shaved area, unlike the remainder of the mice in the test.

### EXAMPLE 2

The above test (Example 1) was repeated except that the dosage of polyamine was 7.5 mg/kg. Similar results were obtained in that the control resumed normal hair re-growth in the shaved areas, whereas the mice treated with the polyamine did not.

### EXAMPLE 3

Two dogs were treated with (R,R)—(HO)₂DEHSPM (dihydroxydiethylhomospermine) s.c. at a dose of 4.3 mg/kg/d for 20 days. The dogs were Holter-Monitored once weekly for evidence of cardiac rhythm disturbances. A post-drug Holter monitoring was performed 5 days post-drug and hair was shaved at that time to allow for the placing of the leads. Approximately 8 weeks post-drug, it was noted that hair from the last Holter monitoring had not grown back.

### EXAMPLE 4

Several dogs were treated with (R,R) or (S,S)—(HO)₂DEHSPM s.c. twice daily for 13 days. The total daily dose was 4-3 mg/kg. One additional dog served as a saline-treated control animal. All of the dogs were Holter monitored once weekly for evidence of cardiac rhythm disturbances. A post-drug Holter monitoring was performed 3 days post-drug and hair was shaved at that time to allow for the placing of the leads. Approximately 6 weeks post-drug, it was noted that although there was considerable hair regrowth in the control dog, this was not the case with the treated animals. The relative order of hair regrowth was the control dog >(S,S)—(HO)₂DEHSPM dogs>(R,R)—HO₂DEHSPM dogs.

I claim:

1. A pharmaceutical composition in unit dosage form adapted for topical application to a human or non-human mammal to control the growth of hair in the area where applied comprising an amount effective therefor of a polyamine or a salt thereof with a pharmaceutically acceptable acid, said polyamine having the formula: 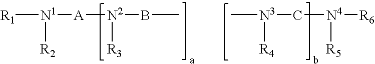
wherein:
R₁-R₆ may be the same or different and are hydrogen, alkyl, hydrocarbyl aryl, hydrocarbyl aryl alkyl, cycloalkyl, or any of the foregoing wherein the alkyl chain is interrupted by at least one etheric oxygen atom with the proviso that R₁ and R₆ may not both be hydrogen;
N¹, N², N³ and N⁴ are nitrogen atoms capable of protonation at physiological pHs;
a and b may be the same or different and are integers from 0 to 6;
A, B and C may be the same or different and are bridging groups of variable length, including unsubstituted heterocyclic bridging groups which incorporate as a hetero atom therein said N¹ or N⁴;
and further wherein said A, B and C are selected such that they effectively maintain the distance between the nitrogen atoms such that the polyamine:
(i) is capable of uptake by a target cell upon topical application of the polyamine to said human or non-human mammal; and
(ii) upon uptake by the target cell, competitively binds via an electrostatic interaction between the positively charged nitrogen atoms to substantially the same biological counter-anions as the intracellular natural polyamines in the target cell; the polyamine, upon binding to the biological counter-anion in the cell, functions to inhibit the formation therein of the enzyme, ornithine decarboxylase.

2. The composition of claim 1 wherein at least one of said A, B and C is a straight or branched chain alkylene group containing at least one —CH(OH)— group which is not alpha- to either of the nitrogen atoms.

3. The composition of claim 1 wherein A, B and C are hydrocarbyl.

4. The composition of claim 3 wherein said hydrocarbyl groups are selected from the group consisting of straight and branched chain alkylene, alkenyl and alkynyl groups, aryl and arylene groups, and cycloalkylene and cycloalkenyl groups.

5. The Composition of claim 1 wherein said polyamine is N,N′-diethylhomospermine, (R,R)-dihydroxydiethylhomospermine or (S,S)-dihydroxydiethylhomospermine.

6. A method for controlling the growth of hair on a human or non-human mammal comprising topically applying to an area of said mammal in which it is desired to regulate the growth of hair an effective amount of the composition of claim 1.

7. A method for controlling the growth of hair on a human or non-human mammal comprising topically applying to an area of said mammal in which it is desired to regulate the growth of hair an effective amount of the composition of claim 2.

8. A method for controlling the growth of hair on a human or non-human mammal comprising topically applying to an area of said mammal in which it is desired to regulate the growth of hair an effective amount of the composition of claim 3.

9. A method for controlling the growth of hair on a human or non-human mammal comprising topically applying to an area of said mammal in which it is desired to regulate the growth of hair an effective amount of the composition of claim 4.

10. A method for controlling the growth of hair on a human or non-human mammal comprising topically applying to an area of said mammal in which it is desired to regulate the growth of hair an effective amount of the composition of claim 5.
